(19) United States
(12) Patent Application Publication
(10) Pub. No.: US 2025/0216385 A1
(43) Pub. Date: Jul. 3, 2025

(54) Low Mass Antigen Detecting Device

(71) Applicant: Korea electronics Technology Institute, Seongnam-si (KR)

(72) Inventors: Kun Nyun KIM, Yongin-si (KR); Yeon Hwa KWAK, Seoul (KR); Min Ji JEON, Seongnam-si (KR); Seung Min MYEONG, Gimcheon-si (KR); Jun Sub KIM, Seoul (KR)

(21) Appl. No.: 18/396,697

(22) Filed: Dec. 27, 2023

(30) Foreign Application Priority Data
Dec. 27, 2022 (KR) 10-2022-0185693

(51) Int. Cl. G01N 33/543; G01N 29/036; G01N 29/24

(52) U.S. Cl. CPC G01N 33/5438; G01N 29/036; G01N 29/2437; G01N 2291/02466

(57) ABSTRACT

Provided is a low mass antigen detecting device for quickly and conveniently detecting various types of antigens. In a low mass antigen detecting device for detecting a low mass antigen through a sensor, the sensor includes a piezo-electric substrate, a sensing layer coupled to one surface of the piezo electric substrate and comprising at least one antigen-BSA, an input IDT electrode disposed at one side of the piezo-electric substrate to apply an input frequency provided as a sweep frequency, and an output IDT electrode disposed at the other side of the piezo-electric substrate to output an output frequency.

### CROSS-REFERENCE TO RELATED APPLICATION

This application claims priority to and the benefit of Korean Patent Application No. 10-2022-0185693 filed on Dec. 27, 2022, in the Korean Intellectual Property Office, the entire disclosure of which is incorporated herein by reference.

### BACKGROUND

### 1. Field

Embodiments relate to a low mass antigen detecting device for quickly and conveniently detecting various types of antigens.

### 2. Description of the Related Art

Surface acoustic wave (SAW) sensors are being widely used to measure changes in environmental factors such as temperature sensors, gas sensors, and biosensors. Such an SAW sensor is divided into two types depending on measurement methods. One is the measurement of the changes in resonance frequency changed by external factors, and the other is the measurement of the change in reflection peak measured by reflection from the SAW sensor.

The operating principles of the surface acoustic wave (SAW) sensor that measures the changes in resonant frequency are as follows. Electrical energy of an input radio frequency (RF) is converted into sound energy having surface acoustic waves at an input inter digital transducer (IDT) and then transmitted. If the generated SAW (a piezo-electric effect occurs) passes through the sensing layer, a change in speed of a frequency occurs depending on target detection. The changed surface acoustic wave is converted again into electrical energy at an output IDT, and thus, a changed signal in response to the target detection is detected.

Currently, the sensing principles of the SAW-based biosensors that detect biomaterials are determined by the change in frequency caused by adsorption of the target material with gold deposited on the sensor surface. This is a study in which input/output IDT electrodes is disposed on a piezo-electric substrate LT (LiTaO3 substrate), and the sensing layer made of gold is deposited between the input/output IDT electrodes. Here, because the receptor biomaterials exist in a functionalized state on the gold-deposited portion, the target material may be adsorbed in a sensing channel. The signal detection is determined by observing an oscillation frequency changed due to the adsorption.

The present disclosure is made in association with a Korean national research and development project (research project name: Development of a portable narcotic detector with high sensitivity for professional investigators, project identification number: 2020M3A9E4039220), and a Korean national research and development project (research project name: Development of a highly sensitive real-time human DNA detection sensor system for forensic and criminal investigation, project identification number: 00154694).

### SUMMARY

Aspects of some embodiments of the present disclosure provide a low mass antigen detecting device for quickly and conveniently detecting various types of antigens.

According to some embodiments, in a low mass antigen detecting device for detecting a low mass antigen through a sensor, the sensor includes: a piezo-electric substrate; a sensing layer coupled to one surface of the piezo-electric substrate and comprising at least one antigen-BSA; an input IDT electrode disposed at one side of the piezo-electric substrate to apply an input frequency provided as a sweep frequency; and an output IDT electrode disposed at the other side of the piezo-electric substrate to output an output frequency.

The sensing layer may include a plurality of antigen-BSA different from each other.

At least one antibody corresponding to the antigen-BSA may be contained in a reagent applied to the sensing layer, and if an antigen corresponding to the antibody is present in a teat target material, binding force of the sensing layer to the piezo-electric substrate may be changed by antigen-antibody binding in the reagent.

The output frequency generated in the output IDT electrode may be classified according to types of antigen-BSA.

If an antigen-antibody reaction corresponding to the antigen-BSA exists in the reagent applied to the sensing layer, the output frequency generated from the output IDT electrode may be continuously output as before application of the reagent.

According to some embodiments, in a low mass antigen detecting device including a cartridge configured to accommodate the sensor, the cartridge includes: a plate on which the sensor is seated; a sidewall protruding from an edge of one surface of the plate; and a connection part protruding from the other surface of the plate and electrically connected to the sensor.

The connection part may be provided as a connector including a pogo pin and configured to allow the cartridge to be coupled to or separated from an external device.

The low mass antigen detecting device may further include a cover configured to cover an upper portion of the sidewall, wherein the cover may be configured to expose a sensing layer of the sensor.

The cover may be provided with a liquid injection hole having a diameter that gradually decreases downward from a surface thereof.

The cover may be physically coupled to the sidewall.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain principles of the present disclosure. In the drawings:

FIG. 1 illustrates a configuration of a sensor for measuring conductivity and changes in frequency in a low mass antigen detecting device according to embodiments;

FIG. 2 illustrates a cross-sectional view of the sensor for measuring the conductivity and the changes in frequency in the low mass antigen detecting device according to embodiments;

FIG. 3 illustrates a view for explaining a principle in which a frequency is changed according to reaction of a specific antigen and an antibody in the low mass antigen detecting device according to embodiments.

FIGS. 4A and 4B illustrate views of results of frequency changes according to the reaction of the specific antigen and the antibody in the low mass antigen detecting device according to embodiments.

FIGS. 5A to 5C illustrate views of a fundamental configuration of the cartridge in the low mass antigen detecting device according to embodiments; and

FIG. 6 illustrates a view of a configuration in which a cover is coupled to the cartridge in the low mass antigen detecting device according to embodiments.

### DETAILED DESCRIPTION

Hereinafter, embodiments will be described in detail with reference to the accompanying drawings.

Embodiments of the present disclosure may, however, be embodied in many different forms and should not be construed as being limited to the embodiments set forth herein; rather, these embodiments are provided so that those skilled in the art thoroughly understand the present disclosure. Rather, these embodiments are provided so that this disclosure will be thorough and complete, and will fully convey the scope of the present disclosure to those skilled in the art.

In addition, in the following drawings, the thickness or size of each layer is exaggerated for convenience and clarity of description, and the same reference numerals in the drawings refer to the same elements. As used herein, the term “and/or” includes any and all combinations of one or more of the associated listed items. In this specification, it will also be understood that if a member A is referred to as being connected to a member B, the member A can be directly connected to the member B or indirectly connected to the member B with a member B therebetween.

The terms used in this specification are for illustrative purposes of the present disclosure only and should not be construed to limit the meaning or the scope of the present disclosure. As used in this specification, a singular form may, unless definitely indicating a particular case in terms of the context, include a plural form. Also, the expressions “comprise” and/or “comprising” used in this specification neither define the mentioned shapes, numbers, processes, operations, members, elements, and/or groups of these, nor exclude the presence or addition of one or more other different shapes, numbers, processes, operations, members, elements, and/or groups of these, or addition of these. The term “and/or” used herein includes any and all combinations of one or more of the associated listed items.

As used herein, terms such as “first,” “second,” etc. are used to describe various members, components, areas, layers, and/or portions. However, it is obvious that the members, components, areas, layers, and/or portions should not be defined by these terms. The terms do not mean a particular order, up and down, or superiority, and are used only for distinguishing one member, component, region, layer, or portion from another member, component, region, layer, or portion. Thus, a first member, component, region, layer, or portion which will be described may also refer to a second member, component, region, layer, or portion, without departing from the teaching of the present disclosure.

Spatially relative terms, such as “below”, “beneath”, “lower”, “above”, “upper” and the like, used herein for ease of description to describe one element or feature's relationship to another element(s) or feature(s) as illustrated in the figures. These spatially relative terms are intended for easy comprehension of the prevent invention according to various process states or usage states of the prevent invention, and thus, the present disclosure is not limited thereto. For example, an element or feature shown in the drawings is turned inside out, the element or feature described as “beneath” or “below” may change into “above” or “upper”. Thus, the term “below” may encompass the term “above” or “below”.

Hereinafter, a fundamental configuration and principle of a sensor constituting a low mass antigen detecting device according to embodiments will be described.

In the low mass antigen detecting device according to embodiments, one configuration of the sensor may be provided as a sensing layer made of a two-dimensional nanomaterial containing antibodies, and a piezo-electric substrate provided as a surface acoustic wave element and a field effect transistor may be coupled to manufacture the sensor to detect liquid biomaterials with high speed, high sensitivity, and reliability.

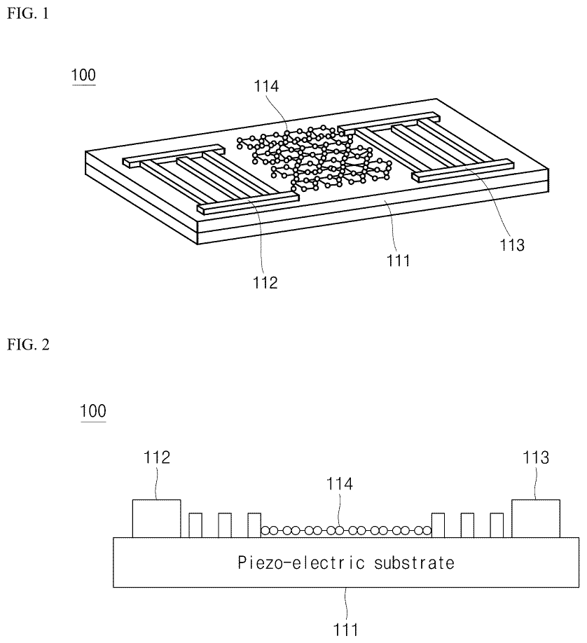

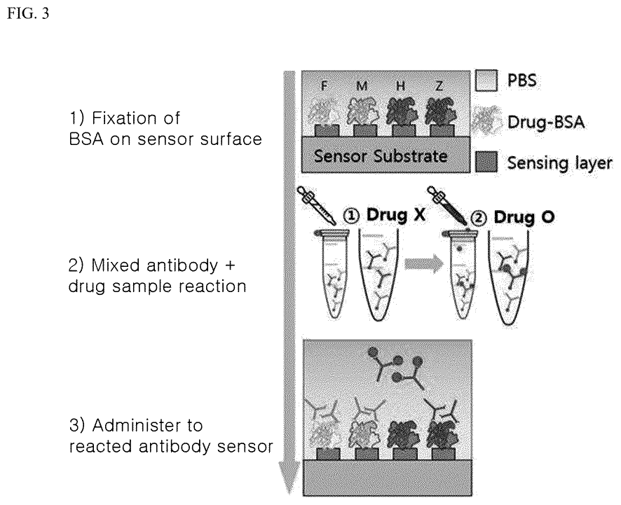

FIG. 1 illustrates a configuration of a sensor for measuring conductivity and changes in frequency in a low mass antigen detecting device according to embodiments. FIG. 2 illustrates a cross-sectional view of the sensor for measuring the conductivity and the changes in frequency in the low mass antigen detecting device according to embodiments.

Referring to FIGS. 1 and 2, specifically, a sensor 100 constituting a low mass antigen detecting device may include a piezo-electric substrate 111, a pair of interdigital transducer (IDT) electrodes 112 and 113 disposed on both ends of the piezo-electric substrate 111, respectively, and a sensing layer 114 between the IDT electrodes 112 and 113.

In some embodiments, the piezo-electric substrate 111 may be configured to generate a potential difference between both the ends if a physical pressure is applied according to an piezo-electric effect.

In some embodiments, the input IDT electrode 112 and the output IDT electrode 113 may be coupled to both the ends of the piezo-electric substrate 111, respectively. In some embodiments, if a sweep frequency f_{sweep} is applied to the input IDT electrode 112, an output frequency fₒᵤₜ at the output IDT electrode 113 may be different from the sweep frequency f_{sweep} due to a surface acoustic wave generated in the piezo-electric substrate 111.

In some embodiments, the sensing layer 114 may be disposed on a top surface of the piezo-electric substrate 111. In some embodiments, the sensing layer 114 may include an antigen-bovine serum albumin (BSA) for a plurality of specific antigens (e.g., specific drug ingredients). In some embodiments, if an antibody reacting with a specific antigen is injected into the sensing layer 114, binding force of the antibody to the surface of the piezo-electric substrate 111 may be changed due to the reaction with the antigen-BSA to affect the surface wave on the piezo-electric substrate 111. In some embodiments, changes in the sweep frequency f_{sweep} of the input IDT electrode 113 and the output frequency fₒᵤₜ of the output IDT electrode 114, which vary depending on the surface wave, may be analyzed to detect whether a specific antigen is present. These mechanism will be described later.

Hereinafter, a detection operation of the sensor in the low mass antigen detecting device according to embodiments will be described in more detail.

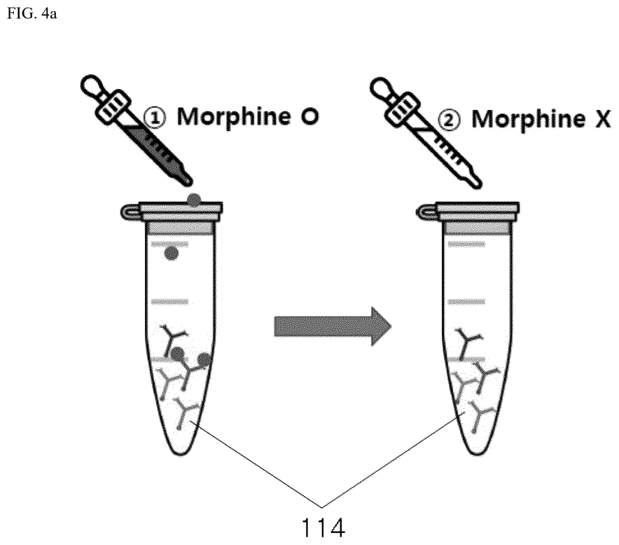

FIG. 3 illustrates a view for explaining a principle in which a frequency is changed according to the reaction of the specific antigen and the antibody in the low mass antigen detecting device according to embodiments.

Referring to FIG. 3, first, 1) the sensing layer may be made of the antigen-BSA (illustrated as four types of F, M, H, and Z in FIG. 3) for a plurality of antigens, which are drug ingredients, on the surface of the piezo-electric substrate.

In some embodiment, 2) a reagent may be formed by mixing a plurality of antibodies (also illustrated as four types of antibodies) corresponding to the plurality of antigens, and a test target material may be injected into the reagent. In 2) of FIG. 3, a comparison between a case in which the test target material does not contain antigens corresponding to the four types of antibodies and a case in which the test target material contains the antigens is illustrated. In some embodiments, if the test target material contains an antigen corresponding to any one of the four types of antibodies, the test target material may react with the corresponding antibody in the reagent (antigen-antibody reaction is illustrated as a red color in FIG. 3).

As a result, as illustrated in 3), when the reagent mixed with the test target material is injected into the sensing layer, if the test target material does not contain a specific antigen, there is no antigen-antibody reaction within the reagent, and thus, there is no difference in binding force of the sensing layer containing the antigen-BSA. In some embodiments, as described above, there is no change in surface acoustic wave on the piezo-electric substrate before and after the reaction, and thus, the output frequency fₒᵤₜ may be significantly changed, and the output frequency fₒᵤₜ may appear discontinuously.

In some embodiments, when the reagent mixed with the test target material is injected into the sensing layer, if a specific antigen is contained in the test target material, antigen-antibody binding may occur, and thus, the binding force of the sensing layer may be weakened by the reaction between the reacting antibody and the antigen-BSA. In some embodiments, because the binding force to the lower piezo-electric substrate is weakened, the surface acoustic waves in the piezo-electric substrate may be weakened, and the output frequency fₒᵤₜ may not be changed significantly, and thus, the output frequency fₒᵤₜ may appear continuously.

In some embodiments, as described above, if the antibody bound to the specific antigen is injected into the sensing layer 114 of FIG. 2 containing the antigen-BSA for the specific antigen, the binding force of the sensing layer 114 to the piezo-electric substrate 111 may be weakened by the reaction, and thus, the change in the output frequency fₒᵤₜ may become relatively small. As a result, if the change in the output frequency fₒᵤₜ is examined, for example, whether the output frequency fₒᵤₜ is continuous or discontinuous is examined, whether the specific antigen is present in the reagent to be tested may be easily detected.

Hereinafter, an example of frequency changes depending on the presence or absence of the antigen in the low mass antigen detecting device according to an embodiment of the present invention will be described.

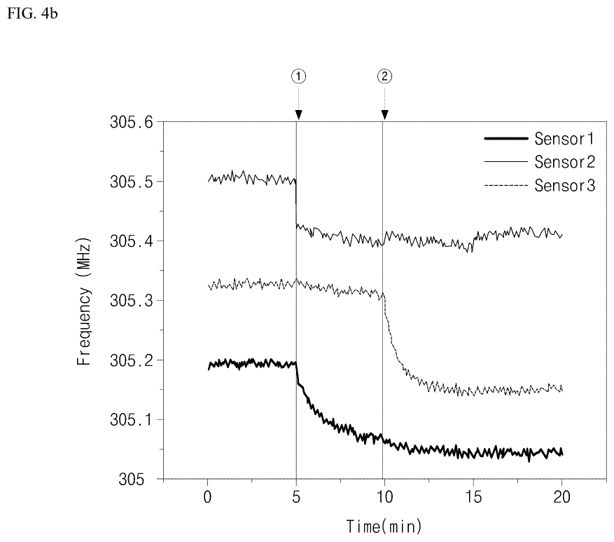

FIGS. 4A and 4B illustrate views of results of frequency changes according to the reaction of the specific antigen and the antibody in the low mass antigen detecting device according to embodiments.

As illustrated in FIG. 4A, a case \textcircled{1} in which a test target material containing a morphine antigen is mixed with a reagent containing four types of specific antibodies, for example, an antibody against morphine and a case \textcircled{2} in which a test target material containing morphine that does not contain the morphine antigen is mixed may be compared to each other. In some embodiments, in the case \textcircled{1} in which the morphine antigen is contained, it may be confirmed that the morphine antigen-antibody reaction occurs, and in the case \textcircled{2} in which the morphine antigen is not contained, it may be confirmed that the antigen-antibody reaction does not occur.

In some embodiments, referring to FIG. 4B, results obtained by injecting each of the reagent \textcircled{1} that contains the morphine antigen and the reagent \textcircled{2} that does not contain the morphine antigen into the sensing layer 114 containing the antigen-BSA at different times are illustrated.

A change in frequency corresponding to the number of antibodies constituting the sensing layer 114 may be observed. For example, the sensing layer 114 in FIG. 4B may be configured to contain three types of antibodies, which contains the aforementioned morphine, and the change in frequency corresponding to each antibody may be indicated as sensor 1, sensor 2, and sensor 3, respectively. In some embodiments, in the difference in binding force depending on the types of respective antibodies, because a difference in surface acoustic wave on the piezo-electric substrate occurs, if the sweep frequency f_{sweep} is applied, graphs may be distinguished to be separated for each type of antibody.

In some embodiments, in the graph of FIG. 4B, if the reagent \textcircled{1} that contains the morphine antigen is injected into the sensing layer 114 at a specific time, is may be seen that, in the other two graphs, the difference in frequency change appears sharply, resulting in a discrete section in frequency, but the graph of the sensor 3, which is indicated in a blue color, is remained to be relatively constant and continuous. In the case of the sensor 3, it may correspond to the antigen-BAS for morphine, and as a result, an experimenter may confirm that the injected reagent contains morphine. In some embodiments, because there is no reaction to the antigen-BSA constituting the remaining two sensors 1 and sensor 2, the experimenter may simultaneously confirm that the corresponding antigen does not exist in the reagent.

In some embodiments, if the reagent \textcircled{2} that does not contain the morphine antigen is injected into the sensing layer 114 at the next specific time (for example, about 10 minutes), the graph of the sensor 3 may also confirm that the discrete section occurs. This may be understood because the antigen-BSA of the sensor 3 also does not react to morphine.

Hereinafter, a configuration of a cartridge to which the sensor 100 is coupled in the low mass antigen detecting device according to embodiments will be described.

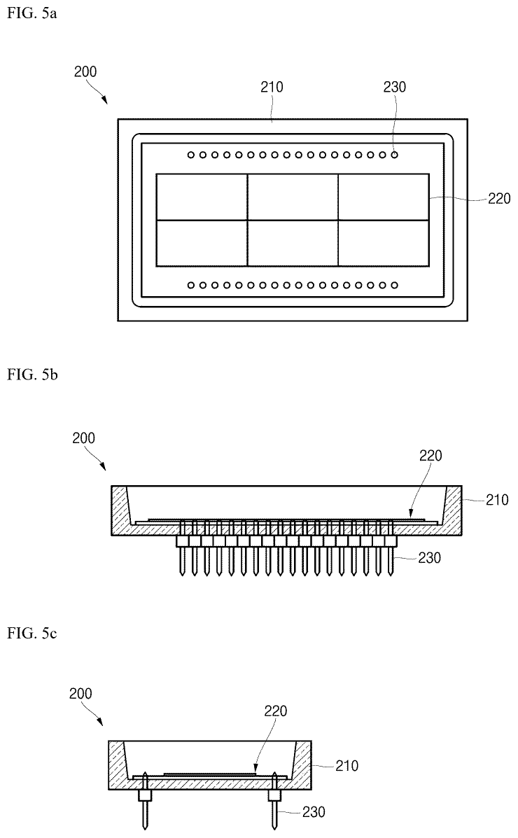

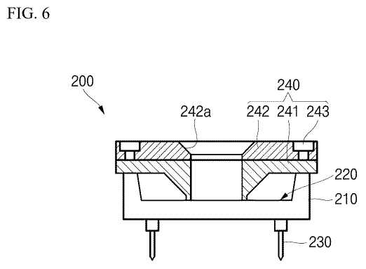

FIGS. 5A to 5C illustrate views of a fundamental configuration of the cartridge in the low mass antigen detecting device according to embodiments. FIG. 6 illustrates a view of a configuration in which a cover is coupled to the cartridge in the low mass antigen detecting device according to embodiments.

First, referring to FIGS. 5A to 5C, a cartridge 200 may be made of a metal material and may include a sidewall 210 constituting a casing, a plate 220 on which a sensor 100 is mounted, and a connection part 230 protruding downward from the plate 220.

The sidewall 210 may protrude in a vertical direction to surround approximately four sides of the plate 220. The sidewall 210 may serve as a casing to protects the sensor 100 disposed therein from an external shock, etc.

The plate 220 may define a bottom surface with respect to the sidewall 210, and the sensor 100 may be disposed in a space defined by the sidewall 210 and the plate 220. In some embodiments, the sensor 100 may be seated on a top surface of the plate 220.

In some embodiments, the connection part 230 may be disposed below the plate 220 and may be electrically connected to the sensor 100 through the plate 220. The connection part 230 may have a shape of a connector constituted by a plurality of pogo pins as illustrated in the drawings, but may also be replaced with a wired cable or in a wireless connection manner depending on selection. In some embodiments, the connection part 230 may allow a structure of the cartridge 200 including the sensor 100 to be connected to a detecting device (not shown) through a separate PCB or directly.

The connection part 230 may input a sweep frequency f_{sweep} generated from the detecting device to the sensor 100 and, conversely, may serve to transmit an output frequency fₒᵤₜ to the detecting device. In some embodiments, the detecting device may be able to detect whether a specific antigen is present in a reagent by checking the presence or absence of an antibody that reacts with the antigen in the sensor 100.

Referring to FIG. 6, a cover 240 may be further coupled to an upper portion of the sidewall 210. The cover 240 may be configured to protect the sensor 100 mounted on the plate 220 and may be configured to surround the sensor 100 except for an area onto which the reagent will be injected. In some embodiments, the cover 240 may include an inner cover 241 that has a hole therein to expose a sensing layer 114 of the sensor 100, and an outer cover 242 provided outside the inner cover 241 and having a liquid injection hole 242a corresponding the hole of the inner cover 241. Although the inner cover 241 and the outer cover 242 are illustrated as separate components, the inner cover 241 and the outer cover 242 may also be molded in one body.

In the case of the liquid injection hole 242a of the outer cover 242, the liquid injection hole 242a may have an inclined surface, which has a large upper diameter and a diameter gradually decreases downward. Thus, if the reagent is injected, the reagent may be transferred to the sensing layer 114 of the sensor 100 along the inclined surface.

In some embodiments, the inner cover 241 and the outer cover 242 may be coupled to the sidewall 210 of the cartridge 200 through a coupling part 243 such as a bolt. In order for the bolt coupling of the coupling part 243, the coupling holes may be formed in advance at corresponding positions of the sidewall 210. In some embodiments, the coupling part 243 such as the bolt may be coupled to pass through the outer cover 242, the inner cover 241, and the sidewall 210 through physical force from the outside without a separate coupling hole.

In the case of the low mass antigen detecting device having the cartridge 200, the cartridge 200 may be easily connected or separated by connecting or disconnecting the connection part 230. Therefore, for example, if a specific drug ingredient is suspected at the scene of an incident at which the police are dispatched, the cartridge 200 having the antigen-BSA for the drug ingredient is combined, and the test may be performed immediately at the scene to easily and accurately test the drug ingredient, thereby determining the presence or absence of the drug ingredient.

In the low mass antigen detecting device according to the embodiments, the sensor may be provided through the sensing layer including the various types of antigen-BSA, and the test target material may be mixed with the reagent composed of various types of antibodies and then be applied to the sensor to easily detect the various types of low mass antigens.

In some embodiments, in the low mass antigen detecting device, the various types of cartridges may be changed to be suitable for the situations in the fields through the structure that accommodates the sensor in the cartridge, thereby detecting the more variety of antigens.

The above-mentioned embodiment is merely an embodiment of the low mass antigen detecting device according to the present invention, and thus, the present invention is not limited to the foregoing embodiment, and also it will be understood by those of ordinary skill in the art that various changes in form and details may be made therein without departing from the spirit and scope of the present invention as defined by the following claims.

What is claimed is:

1. A low mass antigen detecting device for detecting a low mass antigen through a sensor,
wherein the sensor comprises:
a piezo-electric substrate;
a sensing layer coupled to one surface of the piezo-electric substrate and comprising at least one antigen-BSA;
an input IDT electrode disposed at one side of the piezo-electric substrate to apply an input frequency provided as a sweep frequency; and
an output IDT electrode disposed at the other side of the piezo-electric substrate to output an output frequency.

2. The low mass antigen detecting device of claim 1, wherein the sensing layer comprises a plurality of antigen-BSA different from each other.

3. The low mass antigen detecting device of claim 1, wherein at least one antibody corresponding to the antigen-BSA is contained in a reagent applied to the sensing layer, and
if an antigen corresponding to the antibody is present in a teat target material, binding force of the sensing layer to the piezo-electric substrate is changed by antigen-antibody binding in the reagent.

4. The low mass antigen detecting device of claim 1, wherein the output frequency generated in the output IDT electrode is classified according to types of antigen-BSA.

5. The low mass antigen detecting device of claim 1, wherein, if an antigen-antibody reaction corresponding to the antigen-BSA exists in the reagent applied to the sensing layer, the output frequency generated from the output IDT electrode is continuously output as before application of the reagent.

6. A low mass antigen detecting device comprising a cartridge configured to accommodate the sensor of claim 1,
wherein the cartridge comprises:
a plate on which the sensor is seated;
a sidewall protruding from an edge of one surface of the plate; and
a connection part protruding from the other surface of the plate and electrically connected to the sensor.

7. The low mass antigen detecting device of claim 6, wherein the connection part is provided as a connector comprising a pogo pin and configured to allow the cartridge to be coupled to or separated from an external device.

8. The low mass antigen detecting device of claim 6, further comprising a cover configured to cover an upper portion of the sidewall,
wherein the cover is configured to expose a sensing layer of the sensor.

9. The low mass antigen detecting device of claim 8, wherein the cover is provided with a liquid injection hole having a diameter that gradually decreases downward from a surface thereof.

10. The low mass antigen detecting device of claim 8, wherein the cover is physically coupled to the sidewall.
